(19) United States
(12) Patent Application Publication
(10) Pub. No.: US 2024/0273740 A1
(43) Pub. Date: Aug. 15, 2024

(54) Alignment Using Two-Part Marker and Image Visible Marker

(71) Applicant: Novarad Corporation, Provo, UT (US)

(72) Inventors: Wendell Arlen Gibby, Mapleton, UT (US); Steven Todd Cvetko, Draper, UT (US)

(21) Appl. No.: 18/169,800

(22) Filed: Feb. 15, 2023

(51) Int. Cl. G06T 7/33; G06T 11/00; G16H 30/20

(52) U.S. Cl. CPC G06T 7/33; G06T 11/00; G16H 30/20; G06T 2210/41

(57) ABSTRACT

Technology is described to enable alignment of an image data set with an anatomy of a person using an augmented reality (AR) headset. The device may include an image visible marker that is detectable by a medical imaging device and an optical marker. A common connector can removably join the image visible marker to the optical marker and define a fixed distance between the image visible marker and the optical marker.

### BACKGROUND

Mixed or augmented reality is an area of computing technology where images and views from the physical world and virtual computing worlds may be combined into a mixed reality world or view. In mixed reality, views of people, places, and objects from the physical world and virtual constructs become a blended visual environment. A mixed reality experience may be provided through existing commercial or custom software along with the use of VR (virtual reality) or AR (augmented reality) headsets or devices.

Augmented reality (AR) is an example of mixed reality where a live direct view (i.e., a real view) or an indirect view of a physical, real-world environment is augmented or supplemented by computer-generated sensory input such as images, sound, video, graphics or other data. Such augmentation may be performed as a real-world location is viewed and in context with environmental elements. With the help of extended AR technology (e.g. adding computer vision and object recognition) the information about the surrounding real world of the user may become interactive and may be digitally modified (e.g. via computer graphic overlays).

An issue faced by AR systems or AR headsets is identifying a position and orientation of a physical object with a high degree of precision. Similarly, aligning the position of a virtual element with a live view of a real-world environment may be challenging. For example, aligning a virtual object with a physical object as viewed through an AR headset may be difficult because the luminescence or light of the virtual object tends to obscure the underlying physical object with which the virtual object is to be aligned, as viewed by the user. Providing an approximate alignment of a virtual object with a physical object to within a few centimeters may be useful for entertainment, education and less demanding applications but greater positioning and alignment resolution for AR systems may be desired in the scientific, engineering and medical disciplines. As a result, positioning and alignment processes for AR used in the scientific, engineering and medical disciplines may be done manually which can be time consuming, cumbersome, and inaccurate.

### BRIEF DESCRIPTION OF THE DRAWINGS

FIG. 1 illustrates an example of a two-part device to enable alignment of an image data set with anatomy of a person using an augmented reality (AR) headset.

FIG. 2A illustrates an example of a multi-faceted cap for a two-part device to enable alignment of an image data set with anatomy of a patient.

FIG. 2B illustrates an example of a base of a two-part device to enable alignment of an image data set with anatomy of a patient using an augmented reality (AR) headset.

FIG. 3 illustrates one example of a reverse side of a multi-faceted cap as shown in FIG. 2A.

FIG. 4A illustrates an example metal square that may be an image visible marker in the two-part device.

FIG. 4B illustrates an example of an image visible marker that may be visible in an MRI scan.

FIG. 5 illustrates a reverse side of a multi-faceted cap with a common connector to hold or align an image visible code (e.g., metal square) that has an optical marker formed in the material.

FIG. 6 illustrates an example top view of a two-part device with an image visible marker and an optical marker joined by a common connector.

### DETAILED DESCRIPTION

Reference will now be made to the examples illustrated in the drawings, and specific language will be used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

A technology is provided for using an augmented reality (AR) headset to identify one or more optical markers or optical codes (e.g., an AprilTag, QR code, or 2D optical bar code) on a two-part device that has an image visible marker. The two-part device may include an image visible marker that is detectable by a medical imaging device. The two-part device may be placed on a person when a medical imaging device is used to capture a medical image (e.g., a CT (computed tomography) scan, MRI (magnetic resonance imaging) scan or X-ray) of the person. Then the two-part device may be in view of a camera of the AR headset during a medical procedure. The two-part device may enable alignment of an image data set captured by a medical imaging device with an anatomy of a person while medical personnel (e.g., a doctor) are using an augmented reality (AR) headset. The optical marker may a marker such as colored marker, a 2D bar code or an infrared marker. Further, a common connector may removably join the image visible marker to the optical marker. The common connector may define a direction and/or a fixed distance between the optical marker and the image visible marker.

In further example, the two-part device may have a base portion with a layer of material that can be attached to a patient using an adhesive or other physical attachment devices. The base may be made of plastic, fabric, rubber, neoprene or another material. The base may be attached to at least one of: the skin, inner tissue (directly when visible or indirectly using a pin), bone (directly when visible or indirectly using a pin), anatomical landmarks (e.g., the iliac crest, tip of the nose, etc.) or any other anatomical structure of the patient. The base may have an image visible marker attached onto the base and the image visible marker may be joined to the optical marker or the second portion of the device using a common connector. Further, the image visible marker may be attached (e.g., glued or affixed) to the base at any point on the base that known in advance. For example, the image visible marker may be attached on the surface of the base. Alternatively, the image visible marker may be in a protrusion or plastic bump that extends upward from the base.

An additional part of the device may be a cap with one or more facets that can be fitted or joined together to the image visible code on the base using a common connector. A multi-faceted cap may include one or more optical codes on the multi-faceted cap. The multi-faceted cap may have optical codes that are: adhered to the multi-faceted cap, etched into the multi-faceted cap, embossed on the multi-faceted cap, displayed using an e-ink screen on the cap or any other way of enabling the multi-faceted cap to display the optical codes to an AR headset. In one example, one optical code may be on each face of the multi-faceted cap. The multi-faceted cap may be fitted or joined together with the image visible code using a common connector with a void that has one or more walls formed on the bottom (or opposite side) of the multi-faceted cap with the image visible marker on the base.

The image visible code on a base may be joined together with the multi-faceted cap in one or more ways using the image visible code, the image visible code with a covering, a protrusion, a projection or a bump that is inserted or fitted into the common connector on the bottom of the multi-faceted cap. Examples of the joining or fitting structures may include at least one of: a snap fit structure, a press fit structure, spring loaded clips, latches, Velcro or adhering the two parts together.

An image data set can be aligned with a body of a person using one or more optical codes on the cap as coupled or joined to the image visible markers and the two-part device can be located on the body of the person. The image data set may be a previously acquired image of a portion of body of a person using a non-optical imaging modality. Examples of the non-optical imaging modalities may be an MRI (magnetic resonance imaging) modality, CT (computed tomography) scanning modality, X-ray modality, Positron Emission Tomography (PET) modality, an ultrasound modality, a fluorescence modality, an Infrared Thermography (IRT) modality, 3D Mammography, or a Single-Photon Emission Computed Tomography (SPECT) scan modality, etc. The image data set can be aligned to the body of the person using an image visible marker that is a fixed distance from (e.g., to the side of or underneath) at least one optical marker (e.g., optical code) on the cap located on the body or anatomy of the person. The cap may be a multi-faceted cap. For example, an image visible marker and an optical code may be a known distance from each other (e.g., in fixed proximity of each other) to facilitate the alignment of the image data set with the body of the person. An image visible marker is a marker that can be viewed in a non-visible imaging modality, such as a captured radiology image or an image data set, which may not be optically visible to the AR headset.

The image data set may be captured with a representation of the image visible marker in machine captured images that capture structures of the human body with a non-optical imaging modality. The representation of the image visible marker in the image data set may be aligned with the body of the patient using the known fixed position of the image visible marker with respect to the one or more optical markers (e.g., optical codes) on the body of the person using the two-part device (as described in further detail later) that is viewable by an AR headset. For example, the image visible marker may be an enclosed container (e.g., an MRI bead or sphere) on a base or in a protrusion on the base that has a height. Details for using markers, optical codes and image visible markers for alignment of image data sets and images with a body of a person are described further in U.S. Pat. Nos. 11,287,874 to Gibby entitled “USING OPTICAL CODES WITH AUGMENTED REALITY DISPLAYS”; 10,825,563 to Gibby entitled “ALIGNING IMAGE DATA OF A PATIENT WITH ACTUAL VIEWS OF THE PATIENT USING AN OPTICAL CODE AFFIXED TO THE PATIENT”; and 10,010,379 to Gibby entitled “AUGMENTED REALITY VIEWING AND TAGGING FOR MEDICAL PROCEDURES”, which are incorporated by reference in their entirety herein.

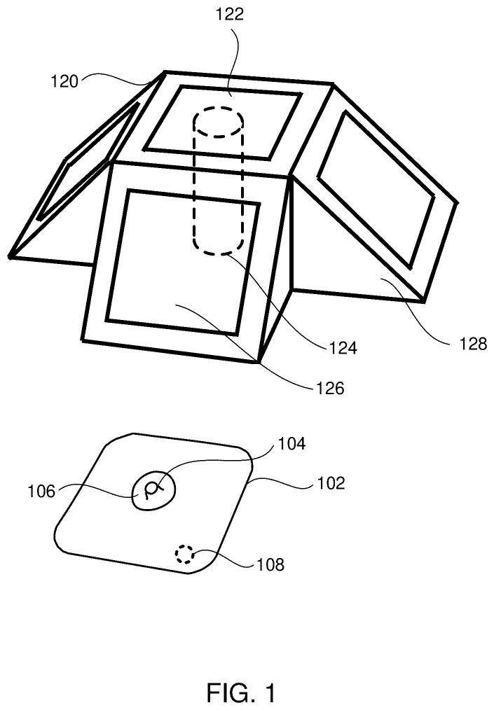

FIG. 1 illustrates a device to enable alignment of an image data set with anatomy of a person using an augmented reality (AR) headset. The device may include a base 102 with an image visible marker 104 that is detectable by a medical imaging device. The base 102 may have the image visible marker 104 on a first surface of the base. Alternatively, the base 102 may have the image visible marker 104 with covering material 106 over the image visible marker 104 on the base 102.

An adhesive layer may be placed on a second surface (or side opposite the image visible marker 104) of the base 102 to enable the base 102 to be adhered to a person's body. Other types of attachment mechanisms may be used to attach the base 102 to the person's body. For example, the attachment may be made using: a hooked attachment, a threaded attachment, or an inserted attachment into the skin. Further alternative ways in which the base 102 may be attached to the patient may include a pin, threaded screw, or another mechanical attachment.

A cover 106 may be formed on a first surface of the base and the cover 106 and/or image visible marker 104 may be fitted to a common connector 124 of the multi-faceted cap. The cover 106 can be associated with the image visible marker 104.

In one example, the base may have the image visible marker 108 in a location near to a projection or coupler that connects to the common connector 124, and the relative position and orientation of the image visible marker 108 with respect to the projection or coupler may be known. Alternatively, a projection or coupler may include the image visible marker 104 within the projection or coupler. For example, the image visible marker 104 may be in a portion of the projection or coupler, or the image visible marker may be located on a longitudinal axis of the projection. The base 102 with the attachment material or structure (e.g., the adhesive or pin) may have a larger area or smaller area than the area covered by the faces 126, 122 of the multi-faceted cap.

A multi-faceted cap 120 (e.g., cap) may have an optical marker 122 on a first face of the cap. For example, the optical marker 122 may be a colored marker or an optical code. A common connector 124 may be associated with or attached to a second surface of the cap to receive the image visible marker 104 and cover 106 and form a removable coupling between the base 102 and the multi-faceted cap 120. The common connector 124 may be removably attached to the cap or the common connector may be permanently attached to the cap so the common connector can be removably attached or joined to the image visible marker 104. In one example configuration, the common connector 124 may have a circular wall and an end wall. In other configurations, the common connector 124 may be a cone, pyramid, hemisphere or another shape that can receive the image visible marker 104 and the cover 106, so that the image visible marker 104 can be aligned into or fitted into the common connector 124. A medical professional may join the image visible marker 104 to the common connector 124 of the cap 120 to form a coupling. The common connector 124 may include any type of common attachment, common link, connector, coupler, connective substrate, connective platform, connective device, coupler, shared connector, rod, flange type coupler, or other connectors to connect the cap 120 and the image visible marker 104.

The coupling formed by the image visible marker 104 and the common connector 124 may be at least one of: a snap coupling, a press fit coupling, an adhesive coupling, a pin coupling, a ratcheted coupling, or a latch type coupling. In the case of the snap coupling, a nub formed using the image visible marker 104 or a protrusion from the base 102 with the image visible marker 104 may be slightly larger than a part of the channel in the receiving common connector 124. As a result, the image visible marker with the nub may be snapped (e.g., using enough compressive force) into the common connector.

In a different configuration, the press fit coupling between the base and the multi-faceted cap may simply be made of plastic or metal that can be press fit together by sliding an image visible marker or a protrusion into the common connector 124. An adhesive coupling may contain an adhesive that fixes the image visible marker 104 or a protrusion within the receiving common connector 124. Further, a coupling may have a pin put through a wall of the common connector 124 and the image visible marker to hold them together. A latch style coupling may have a spring-loaded latch and a latch hole which holds the image visible marker 104 in a receiving void of the common connector 124 until the latch is released by the medical professional. A ratcheting connection may have a pawl and ratchet mechanism that may not be releasable or the ratcheting connection may have a releasable pawl.

A second optical code 126 may be located on a second face or facet of the cap 120. The second optical code 126 may be oriented at between a one-degree angle up to an 80-degree angle with respect to the first optical code 122. Accordingly, there may be one or more additional optical codes with faces oriented at a pre-defined angle (e.g., 45 degrees) with respect the first optical code 122. For example, there may be four optical codes on four faces of the multi-faceted cap. The four additional optical codes may be separated from each other radially at 90 degree angles with respect to one another around a center axis of the multi-faceted cap. Where there are greater or fewer facets and optical codes, the degrees for separation between them may be set based on the number of facets that exist. For example, two facets may be 180 degrees apart, three facets may be 120 degrees apart, five facets may 72 degrees apart, six facets may be 60 degrees apart, etc. Alternatively, the facets and optical codes may be spaced at uneven or random degree or measurement intervals.

The shape of the multi-faceted cap can allow a user of an AR headset to see multiple optical codes at the same time. For example, a user or medical professional may be able to see at least two or more optical codes at any time while looking at the multi-faceted cap without having the move around the patient to see more optical codes. Being able to see more optical codes at one time may mean that the user or medical professional has to spend less time registering optical codes with the AR headset, in order to perform the alignment process for an image data set or X-ray generated image with the body of the person or patient.

FIG. 1 illustrates that a 45-degree angle is a useful angle for making the optical codes more easily visible to the user of the AR headset. However, any angle between approximately 1 and 85 degrees may be used to enable the optical codes to be registered quickly and effectively with the AR headset. Optical codes may also be placed on the intermediate or edgewise faces 128 of the main facets.

In yet another configuration of the device, the cap may be in the shape of a cone with the top cut off. In this example, the facet on the side of the cap may be a single angular slope that skirts a top face that is a circle. In this example, the optical codes may be anywhere on the sloped surface that slopes away from the top face and one optical code may be on the top circular face. The top face may be of any geometrical shape (e.g., circle, square, hexagon, octagon, etc.) and each of the faces on the cap may be made in any geometrical or artistic shape.

In one example, the base can include an EKG (electrocardiogram) electrode as the image visible marker 104 on the base. The electrode may have a sticky adhesive on the second surface of the base and may also have a metal or conductive electrode as the image visible marker 104. Thus, an EKG pad or electrode can be on the base with adhesive that is attached to a patient. When the adhesive on the base is attached to the patient then the medical professional can snap a multi-faceted cap with an optical code onto the EKG pad.

The image visible marker 104 may also be an MRI (magnetic resonance imaging) marker. This image visible marker 104 may then be part of the image data set that is captured using the medical imaging machine. The image visible marker 104 may also be a radiopaque marker or a metal optical code and may be in a projection or in proximity to a projection. Thus, at least one image visible marker 104 is provided that is visible on an MRI image, CT scan, etc. Then optical codes 122, 126 can be joined together with the image visible marker 104 using the common connector 124.

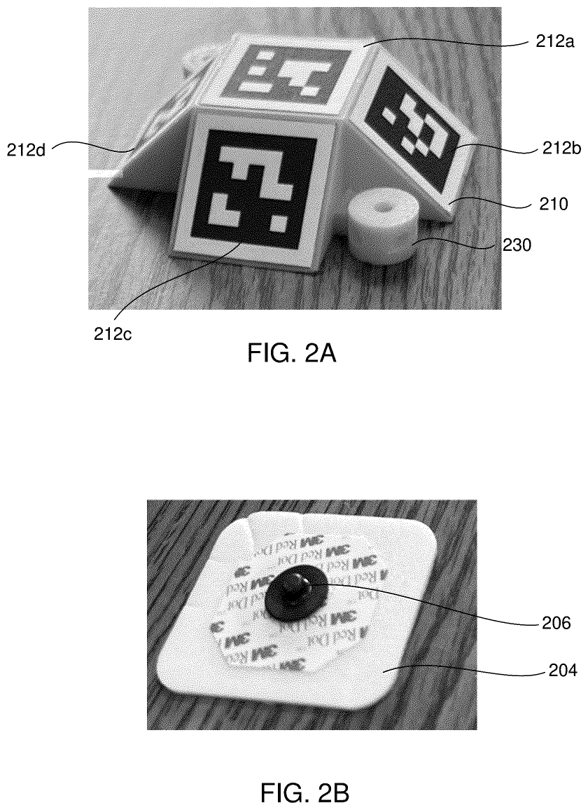

FIGS. 2A and 2B illustrate two parts of a device to enable alignment of an image data set with anatomy of a patient using an augmented reality (AR) headset. FIG. 2A illustrates a cap 210 (e.g., a multi-faceted cap) used with the technology. FIG. 2B illustrates that the device may have a base 204 having an image visible marker 206 that is detectable by a medical imaging device. A snap configuration for the image visible marker 206 is illustrated in FIG. 2B.

An adhesive layer may be located on a second surface or back surface of the base 210 (not shown) to enable the base to be adhered to a person's body. In addition, the image visible marker 206 to be connected to the common connector may be attached to or contained in a structure on a first surface or front surface of the base. For instance, the image visible marker 206 can be co-incident with structure for being joined to the common connector or the image visible marker 206 may be inside a structure that can be connected to the common connector. For example, the image visible marker may be inside a protrusion that is press fit into the common connector or otherwise connected to the common connector.

Returning to FIG. 2A, a multi-faceted cap 210 may include a plurality of optical codes on a plurality of surfaces or faces of the multi-faceted cap 210. The first optical code 212a may be on the first surface or top surface of the multi-faceted cap 210. Additional optical codes 212b, 212c, 212d may be located on additional surfaces or side surfaces of the multi-faceted cap and these faces may be oriented at an angle (in a convex fashion) to the first surface of the multi-faceted cap and out away from the radial axis of the cap. One additional optical code in FIG. 2A on the opposite side of the cap cannot be seen from the viewed perspective. The optical codes on the additional faces or surfaces 212b, 212c, 212dillustrated by FIG. 2A may be at a 45 degree angle with respect to the optical code on the first surface. In other configurations, the optical codes may be at angle anywhere from 1 degree to 85 degrees from the optical code 212a on the first surface of the multi-faceted cap 210. Furthermore, the optical codes on the additional or side surfaces may be separated from one another at 90-degree angles radially with respect to a center axis of the multi-faceted cap and one another. The radial separation may be anywhere between 1 to 179 degrees of separation. The amount of radial separation may be affected by the number optical codes on the side of the multi-faceted cap that are provided. As already discussed, there may be two, four, five or more optical codes on the sides of the multi-faceted cap and the sizes of the optical codes may vary individually or may vary as a group.

A pin anchor hole 230 (FIG. 2A) or attachment anchor hole may be part of the multi-faceted cap. This pin anchor may have a cylindrical hole through which a pin or screw may be placed to anchor the multi-faceted cap to a bone or other tissue of a patient. There may be one or more of the attachment anchors, as desired. For example, a pin anchor may be between a pair of faces for the multi-faceted cap.

The common connector can be formed on a second surface or the back side of the multi-faceted cap. The image visible marker (and any covering or protrusion) on the base can be press fit into the common connector so that the multi-faceted cap and the image visible marker stay aligned with a known amount of distance between them both. In one example, the multi-faceted cap may have a common connector with a compartment or guide with an open side or a void into which the image visible marker can be inserted. For example, the image visible marker may be an MRI marker, a radiopaque marker, a metal marker, a titanium marker with an optical code formed into the titanium marker or an ultrasound marker. The metal marker may be a metal shape that has no markings on the metal marker, such as a circle, square, rectangle, triangle, sphere, a 2D shape, a 3D shape, or another metal shape. In addition, the metal marker may have an optical code or another coding that is formed into the metal. This optical code in the metal may be visible in an X-ray generated image, such as a 2D X-ray or CT scan.

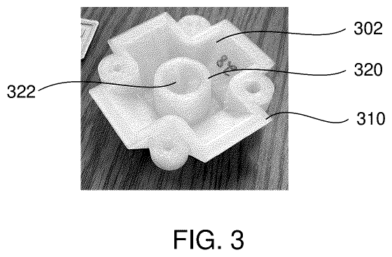

FIG. 3 illustrates one configuration of a reverse side of a multi-faceted cap 302 as compared to the first side or top side of the multi-faceted cap depicted in FIG. 2A. The bottom of the multi-faceted cap may have flat areas 310 which may rest on the body of a person or medical patient. The bottom of the multi-faceted cap may also have one example of a common connector 320 which uses one or more walls to form a void 322 in the underside of the multi-faceted cap. One wall may be circular as illustrated or the walls may form another geometric shape (e.g. square, triangle, hexagon, octagon, irregular shape, etc.) that provide a form fit for the image visible marker or covering for the image visible marker. For example, the void or chamber in the common connector 320 may also be triangular, square, pentagonal, hexagonal, octagonal, etc. In one configuration, the image visible markers may be on the base and may be in a known location under the multi-faceted cap.

Thus, one or more image visible markers may be under the multi-faceted cap at a defined location when the cap is being used for alignment of an image data set to a person's body.

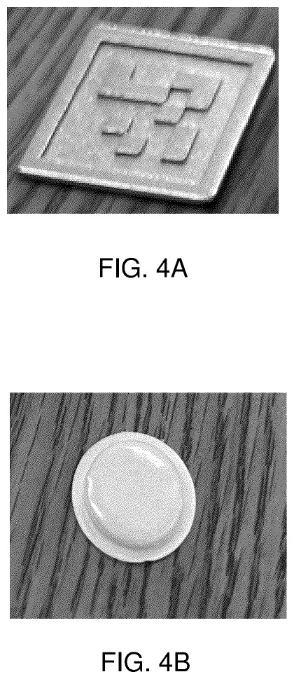

FIG. 4A illustrates a metal square as one configuration of an image visible marker that can be visible in an image data set captured by a medical imaging device. In one example, the image visible marker may be made of titanium or another radiopaque material. The optical code or 2D bar code visible in the metal square of FIG. 4A may also be visible in images captured using other imaging modalities such as X-ray generated images or any other imaging modality where radiopaque materials may be visible. The shape of the common connector on the back of a cap may be square shaped to receive the metal square. The radiopaque image visible marker may be formed in any shape and the shape of the common connector can match the shape of image visible marker. The radiopaque image visible marker may be attached or affixed to the base and then the radiopaque image visible marker may be pressed into the common connector associated with the multi-faceted cap. In this case, the optical codes on the multi-faceted cap may be visible to an AR head set and image visible optical codes may include a 2D bar code that can be recognized in an image data set. For example, the image visible metal code may be visible in an X-ray generated image or a CT scan and can be used to align an image data set of the patient to the patient's physical body using the AR headset.

FIG. 4B illustrates another image visible marker that may be visible in an MRI scan. More specifically, the gel button illustrated may have MR marker material within the plastic container. The gel button may be used when an image data set is captured using MRI imaging and the cap with the optical code can be put on top and aligned with or placed over the gel button. This is a two part structure to provide alignment of an image data set with the patient using the optical code visible to the AR headset and the image visible marker in the image data set. Thus, the optical code and image visible marker can be mechanically referenced using the two-part structure. Alternatively, the image visible marker may be an MR marker that is placed on any location on the base, and there may be a separate snap on the base for connecting the multi-faceted cap to the base.

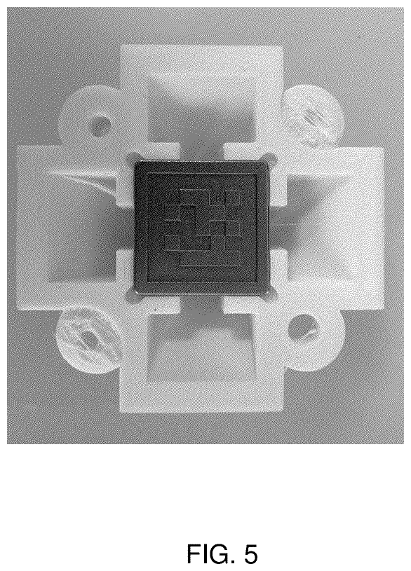

FIG. 5 illustrates a bottom side of a multi-faceted cap with a common connector to hold or align an image visible code (e.g., a metal square) that has an optical marker formed in the material. The common connector that is formed on the bottom side of the multi-faceted cap may use flanges or brackets to hold the image visible code in place.

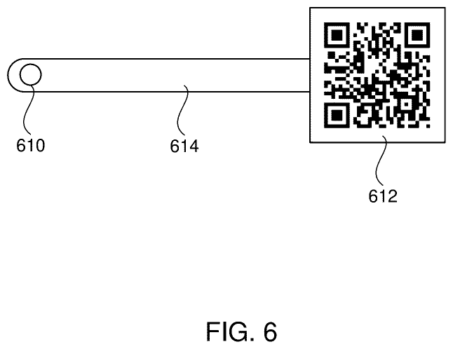

FIG. 6 illustrates an example of a two-part device with an image visible marker 610 and an optical marker 612 joined by a common connector 614. The device may be used to enable alignment of an image data set with an anatomy of a person using an augmented reality (AR) headset. The device may include an image visible marker 610 and the image visible marker 610 may be captured in an image data set of a patient's anatomy using a medical imaging device (e.g., a CT scan, an MRI, etc.) The image visible marker 610 may be detectable and recordable by a medical imaging device.

The device may also include an optical marker 112. The optical marker 112 may be a QR code as illustrated. The optical marker may be any type of optical marker that can be detected by the AR headset. For example, the optical marker may be a shaded or colored shape. In additional examples, the optical marker may be a linear bar code or an April code.

A common connector 614 can be used in the device to removably join the image visible marker 610 to the optical marker 612. The common connector 614 may also define a fixed distance and a direction between the image visible marker and the optical marker.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

1. A device to enable alignment of an image data set with an anatomy of a person using an augmented reality (AR) headset, comprising:
an image visible marker that is detectable by a medical imaging device;
an optical marker; and
a common connector to removably join the image visible marker to the optical marker and to define a fixed distance between the image visible marker and the optical marker.

2. The device as in claim 1, wherein the common connector defines a direction for the fixed distance between the optical marker and the image visible marker.

3. The device as in claim 1, wherein the optical marker is at least one of an optical code, a shaded marker, a colored marker, or an infrared marker.

4. The device as in claim 1, further comprising a base on which the image visible marker is attached.

5. The device as in claim 4, further comprising an adhesive layer on a surface of the base to enable the base to be adhered to a person's body.

6. The device as in claim 4, further comprising a projection formed on a surface of the base, wherein the projection is associated with the image visible marker.

7. The device as in claim 1, further comprising a cap on which the optical marker is presented.

8. The device as in claim 7, further comprising a second optical marker on a second face of the cap.

9. The device as in claim 8, wherein the second optical marker is oriented at a 45-degree angle with respect to the optical marker.

10. The device as in claim 1, further comprising four additional optical markers with faces oriented at a 45-degree angle with respect the optical marker that is a first optical code.

11. The device as in claim 10, wherein the four additional optical markers are oriented radially at 90-degree angles with respect to one another around a center axis of a cap.

12. The device as in claim 4, wherein the common connector is configured to fit a cap to the base and is at least one of: a snap coupling, a press fit coupling, an adhesive coupling, a Velcro coupling, or a pin connected coupling.

13. The device as in claim 4, wherein the base includes an EKG (electrocardiogram) electrode as the image visible marker.

14. The device as in claim 1, wherein the image visible marker is an MRI (magnetic resonance imaging) marker or a radiopaque marker.

15. The device as in claim 14, wherein a projection contains the MRI marker or radiopaque marker.

16. The device as in claim 1, wherein the image visible marker is a metal marker in the shape of at least one of: a circle, square, rectangle, triangle, sphere, a 2D shape, or a 3D shape.

17. A device to enable alignment of an image data set with anatomy of a patient using an augmented reality (AR) headset, comprising:
an image visible marker that is detectable by a medical imaging device;
a plurality of optical codes detectable by the AR headset; and
a common connector to removably join the image visible marker to the plurality of optical codes to define a fixed distance and direction between the image visible marker and the optical codes.

18. The device as in claim 17, further comprising an optical code on a top surface of a multi-faceted cap and optical codes on angled surfaces of the multi-faceted cap.

19. The device as in claim 17, wherein optical codes are on angled side surfaces and are at least a 25-degree angle with respect to the optical code on a top surface of a cap.

20. The device as in claim 17, wherein the optical codes on side surfaces are at 90-degree angles radially with respect to one another and around a center axis of a multi-faceted cap.

21. The device as in claim 17, wherein the common connector is at least one of: a snap coupling, a press fit coupling, or an adhesive coupling.

22. The device as in claim 17, wherein the common connector is configured to receive the image visible marker that is at least one of: an MRI marker, a radiopaque marker, a metal marker, a titanium marker with on optical code formed in the titanium marker, or an ultrasound marker.

23. A device to enable alignment of an image data set with anatomy of a patient using an augmented reality (AR) headset, comprising:
a metal marker that is detectable by a medical imaging device;
a cap having an optical code detectable by the AR headset; and
a common connector to join the optical code to the metal marker to define a fixed distance between the metal marker and the optical code.

24. The device as in claim 23, further comprising an adhesive layer associated with the metal marker to enable the metal marker to be adhered to a person's body.
